(19) United States
(12) Patent Application Publication
(10) Pub. No.: US 2023/0039242 A1
(43) Pub. Date: Feb. 9, 2023

(54) PROVISION OF A THERAPY PLAN

(71) Applicant: Siemens Healthcare GmbH, Erlangen (DE)

(72) Inventors: Alois Regensburger, Poxdorf (DE); Tobias Lenich, Nurnberg (DE)

(21) Appl. No.: 17/881,763

(22) Filed: Aug. 5, 2022

(30) Foreign Application Priority Data
Aug. 5, 2021 (DE) 10 2021 208 498.2

(51) Int. Cl. G16H 20/40; A61N 7/02; G16H 50/50; A61B 34/10

(52) U.S. Cl. CPC G16H 20/40; A61N 7/02; G16H 50/50; A61B 34/10; A61B 2034/104

(57) ABSTRACT

A method for providing a therapy plan includes receiving a dataset. The dataset maps an examination object including a first tissue area at at least one first timepoint. The first tissue area is identified in the dataset as a first state. A second state is specified for the first tissue area. A therapy plan for a therapy apparatus is determined by application of a model to input data. A change in the first tissue area between the at least one first timepoint and at least one second timepoint is createable by the control of the therapy apparatus in accordance with the therapy plan. The model maps the first state to the second state and/or the second state to the first state. The therapy plan is provided as output data of the model.

This application claims the benefit of German Patent Application No. DE 10 2021 208 498.2, filed on Aug. 5, 2021, which is hereby incorporated by reference in its entirety.

### BACKGROUND

The present embodiments relate to a method for provision of a therapy plan, a computer-implemented method for provision of a trained function, a provision unit, a system, and a computer program product.

Frequently, masses in an examination object lead to a pressure load (e.g., squashing) of neighboring tissue (e.g., a hollow organ, such as an artery and/or vein). Masses may further lead to a restriction in movement of the examination object. A mass in this case may describe a tissue area within the examination object, with the tissue area exhibiting a volume increase. The volume increase of the tissue area may have been caused in this case by a beneficial or malignant change (e.g., a tumor) and/or by a cyst and/or another tissue increase (e.g., an obese increase in fat).

For reducing the volume of the mass (e.g., for relieving pressure on adjacent tissue and/or for restoring the movement capability of the examination object), the mass may be surgically resected. Disadvantageously, an open surgical access to the mass on the examination object is often required for this. Further, surrounding tissue may be damaged by the surgical resection.

For treatment of the mass without open surgical access, therapeutic ultrasound in the form of highly intensive focused ultrasound (HIFU) (e.g., histotripsy as a subsidiary type of HIFU) may be employed. In this case, the therapeutic ultrasound may be embodied to destroy at least some areas of tissue in the mass and/or or in regions bordering on the mass (e.g., to lyse the areas of tissue). This enables a volume of the mass to be reduced. Often, the tissue area to be destroyed is specified directly in preoperative and/or intraoperative image data of the examination object. The specification of the tissue area to be destroyed in the preoperative and/or intraoperative image data of the examination object often leads to an unspecific reduction in the mass. In the medium term, this may result in a renewed pressure load on the neighboring tissue and/or to a restriction in movement of the examination object.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, improved planning for a lasting treatment of masses is provided.

Methods and apparatuses for provision of a therapy plan and methods and apparatuses for provision of a trained function are described below. Features, advantages, and alternate forms of embodiment of data structures and/or functions in methods and apparatuses for provision of a therapy plan may be transferred to analogous data structures and/or functions in methods and apparatuses for provision of a trained function. Analogous data structures may be identified, for example, by the use of the prefix “training”. Further, the trained functions used in methods and apparatuses for provision of a training plan may, for example, have been adapted and/or provided by methods and apparatuses for provision of a trained function.

In a first aspect, the present embodiments relate to a, for example, computer-implemented method for provision of a therapy plan. In this case, a preacquired dataset is received. The preacquired dataset maps an examination object including a first tissue area at at least one first point in time. A mapping of the first tissue area in the dataset is further identified as a first state. Further, a second state for the first tissue area is specified. In this case, the second state defines a spatial extent (e.g., a maximum spatial extent) and/or a second tissue parameter of at least one part of the first tissue area at at least one second point in time after the at least one first point in time. A therapy plan for a therapy apparatus is further determined by application of a model to input data. In this case, the input data is based on the dataset and the second state. The therapy plan further has at least one parameter for control of the therapy apparatus. In this case, a change in the first tissue area between the at least one first point in time and the at least one second point in time is able to be created by control of the therapy apparatus in accordance with the therapy plan. The model further simulates a biological growth and the change in the first tissue area between the at least one first point in time and the at least one second point in time. In this case, the model maps the first state to the second state and/or the second state to the first state. After this, the therapy plan is provided as output data of the model.

In this case, the acts of the method for provision of a therapy plan described here may be carried out after one another and/or at least partly simultaneously. The acts of the method may further be computer-implemented at least partly (e.g., completely).

The receipt of the dataset may, for example, include an acquisition and/or readout of a computer-readable data memory and/or a receipt from a data memory unit (e.g., a database). The dataset may further be provided by a provision unit of a medical imaging device for recording the dataset. The medical imaging device for recording the dataset may be configured, for example, as a magnetic resonance tomography installation (MRT) and/or computed tomography installation (CT) and/or medical x-ray device and/or positron emission tomography installation (PET) and/or ultrasound device.

The dataset may have first medical image data that maps the examination object including the first tissue area spatially two-dimensionally (2D) and/or three-dimensionally (3D). The examination object may, for example, be a human and/or animal patient. In this case, the dataset (e.g., the first medical image data) may map the examination object including the first tissue area resolved over time at at least one first point in time (e.g., at a number of first points in time). The dataset (e.g., the first medical image data) may have a number of image points with image values that map the examination object including the first tissue area. Where the dataset maps the examination object including the first tissue area resolved over time, the image points may have time intensity curves. This enables the dataset to map a further change in the examination object (e.g., in the first tissue area; a flow of contrast medium and/or a movement such as a physiological movement). In one embodiment, the dataset may map a vascularization of the examination object (e.g., of the first tissue area). In this case, the vascularization may describe a density of blood vessels and/or lymphatic transport paths in the examination object (e.g., the first tissue area).

In one embodiment, the dataset (e.g., the first medical image data) may be recorded by the medical imaging device at the at least one first point in time (e.g., at the number of first points in time; preacquired). The dataset may further have metadata. The metadata may, for example, include information about a recording parameter and/or operating parameter of the medical imaging device.

The identification of the mapping of the first tissue area may include a segmentation and/or emphasis and/or classification and/or mark (e.g., annotate) image points and/or an image area having a number of image points in the dataset. The identification of the mapping of the first tissue area in the dataset may further take place manually, semi-automatically, or fully-automatically. The first tissue area may include a first anatomical region of the examination object (e.g., a tissue, such as a tumor tissue and/or a mass, and/or an organ, such as a hollow organ, and/or a vessel section, such as an artery and/or vein). Further, the first tissue area may be delimited at least partly (e.g., completely) by a tissue boundary of adjacent tissues. In one embodiment, the tissue boundary may be mapped in the dataset. In this case, the identification of the mapping of the first tissue area may take place with the aid of the tissue boundary.

For example, a graphical display of the dataset may be shown by a display unit. In this case, a medical operator may identify the mapping of the first tissue area in the graphical display of the first tissue area (e.g., through a first input of the medical operator using an input unit). Further, the first input may be associated with the dataset (e.g., the image points of the dataset) as regards the graphical display of the first tissue area (e.g., via a mapping rule for creating the graphical display of the first tissue area from the dataset).

The identification of the mapping of the first tissue area may further be based on a comparison of the image values of the image points of the dataset with a predetermined image value interval and/or a threshold value. As an alternative or in addition, the identification of the mapping of the first tissue area may take place with the aid of geometrical and/or anatomical features of the first tissue area that are mapped in the dataset (e.g., a contour and/or a contrast and/or contrast course, and/or by a comparison with an anatomy atlas). In one embodiment, in the identification of the mapping of the first tissue area, the image points of the dataset which map the first tissue area may be identified.

For semi-automatic identification of the mapping of the first tissue area, the medical operating personnel may, for example, predetermine a point and/or a spatial region within the first tissue area in the graphical display of the first tissue area using the first input. Based on the first input, the mapping of the first tissue area in the dataset may be identified automatically after this (e.g., by a segmentation).

In one embodiment, the identification of the mapping of the first tissue area in the dataset enables a delimited (e.g., a spatially delimited) and/or contiguous image region to be identified, which maps the first tissue area.

The first state may be identified by characteristics (e.g., all characteristics, characteristics of the first tissue area) that the dataset (e.g., the image points) has for the identified mapping of the first tissue area (e.g., the identified image region). The first state may be characterized by a spatial extent (e.g., a shape, such as a surface shape of the tissue boundary) and/or a volume of the first tissue area. In one embodiment, the first state may describe the first tissue area at the at least one first point in time isotropically or anisotropically. The first state may include a virtual representation of the first tissue area at the at least one first point in time (e.g., a volume mesh model) with the virtual representation being defined by the characteristics of the first tissue area (e.g., the spatial extent of the first tissue area described in the dataset).

The specification of the second state may include a specification of a spatial extent (e.g., a maximum spatial extent; of a shape and/or a volume) of at least a part of the first tissue area (e.g., of the entire first tissue area) for the at least one second point in time. In this case, the at least one second point in time may lie temporally after the at least one first point in time (e.g., after the number of first points in time). As an alternative or in addition, the specification of the second state may include a specification of a second tissue parameter (e.g., isotropic or anisotropic) of the at least one part of the first tissue area (e.g., of the entire first tissue area) for the at least one second point in time. The second tissue parameter may, for example, include a mass and/or a vessel infiltration and/or a deformability and/or an elasticity and/or a porosity (e.g., spatially resolved two-dimensionally and/or three-dimensionally). The second state may include a virtual representation of the first tissue area at the at least one second point in time, which is defined by the, for example, maximum spatial extent and/or the second tissue parameter.

The specification of the second state for the first tissue area may take place manually, semi-automatically, or fully-automatically. For a manual and/or semi-automatic specification of the second state, a graphical display of the dataset may be displayed by the display unit. In this case, the first tissue area may be highlighted in the graphical display of the dataset. The medical operating personnel may specify the second state with the aid of the graphical display of the dataset (e.g., in the graphical display of the dataset), for example, through a second input using the input unit. In this case, the medical operating personnel may specify the spatial extent (e.g., maximum spatial extent) and/or the second tissue parameter for the at least one part of the first tissue area for the at least one second point in time completely or partly. The part or complete specification of the second state (e.g., of the spatial extent and/or of the second tissue parameter) may relate to a spatial dimension and/or the spatial extent to be specified and/or the second tissue parameter to be specified. For a part specification of the second state by the medical operating personnel, the definition of the second state for the first tissue area may be completed semi-automatically (e.g., based on the first state). For example, the medical operating personnel may specify the second state semi-automatically with the aid of a two-dimensional graphical display of the dataset. The definition of the second state is completed for the first tissue area three-dimensionally. As an alternative, the second state may be specified based on the dataset (e.g., specified fully-automatically based on the first state of the first tissue area). For this, for example, the first state may be compared with a statistical model, with the statistical model mapping a comparable tissue area. The second state may further be specified such that a deviation between the second state and the statistical model may be minimized.

The therapy plan is determined for the therapy apparatus by application of the model to the input data. In this case, the input data may be based on the dataset (e.g., the first state) and the second state. For example, the input data may include the dataset (e.g., the first state and/or the virtual representation of the first tissue area at the at least one first point in time) and the second state (e.g., the virtual representation of the first tissue area at the at least one second point in time). The model may further provide the therapy plan as output data.

The therapy plan may have at least one parameter (e.g., an operating parameter and/or a positioning parameter) for controlling the therapy apparatus. In this case, the operating parameter may predetermine a physical variable for control of the therapy apparatus (e.g., an amplitude and/or pulse duration and/or current strength and/or voltage and/or temperature). The positioning parameter may further predetermine a spatial position and/or alignment and/or pose of the therapy apparatus (e.g., with regard to the examination object and/or the first tissue area). The therapy plan may include a number of parameters (e.g., a temporal sequence of parameters) for a, for example, step-by-step and/or continuous control of the therapy apparatus. The therapy plan may have the at least one parameter for manual, semi-automatic, or fully automatic control of the therapy apparatus. For a manual or semi-automatic control of the therapy apparatus, the therapy plan (e.g., the at least one parameter of the therapy plan) may have at least one workflow instruction for the medical operating personnel for control and/or positioning of the therapy apparatus. As an alternative, the therapy plan (e.g., the at least one parameter of the therapy plan) may be embodied for a fully-automatic (e.g., robotic) control of the therapy apparatus (e.g., using a provision unit).

If the result of the simulation of the biological growth and the change in the first tissue area is that the second state may only be achieved insufficiently accurately by the change able to be created by the therapy apparatus in the first tissue area, the model for determining the therapy plan may also have a workflow instruction for treatment and/or therapy of the first tissue area and/or of further tissue bordering on the first tissue area.

The change (e.g., a lysis of cells) in the first tissue area between the at least one first point in time and the at least one second point in time may be able to be created by the control of the therapy apparatus in accordance with the therapy plan (e.g., by an input of energy into the first tissue area in accordance with the therapy plan). In this case, the change may be able to be created without surgical access (e.g., open surgical access) in the first tissue area by the control of the therapy apparatus in accordance with the therapy plan. The change may further be able to be created selectively and/or spatially restricted in the first tissue area by the control of the therapy apparatus in accordance with the therapy plan. In one embodiment, the change may be able to be created in the first tissue area at a further point in time or within a period of time between the at least one first point in time and the at least one second point in time by the control of the therapy apparatus in accordance with the therapy plan.

The model may simulate the biological growth of the first tissue area and the change (e.g., therapy-induced change) in the first tissue area between the at least one first point in time and the at least one second point in time (e.g., simultaneously or sequentially). For example, the model may determine the therapy plan including the at least one parameter in such a way that, through the simulation of the biological growth and the change in the first tissue area between the at least one first point in time and the at least one second point in time, the first state is mapped to the second state and/or the second state is mapped to the first state. In this case, the simulation of the biological growth and the change in the first tissue area between the at least one first point in time and the at least one second point in time may include a calculation and/or adjustment and/or approximation of the at least one parameter (e.g., of the number of parameters) of the therapy plan.

The simulation of the biological growth in the first tissue area may be based on the dataset (e.g., the time-resolved mapping of the first tissue area in the dataset). In this case, the dataset may map the biological growth of the first tissue area (e.g., within the first tissue area) on a time-resolved basis at the number of first points in time. In one embodiment, the model may be adjusted based on the dataset. For example, the model may map the first tissue area as a spatial arrangement of volume elements (e.g., isotropic or anisotropic). In this case, the simulation may be based, for example, on a finite element method (FEM). Further, the model may in each case have a growth parameter (e.g., a growth direction and/or a growth speed and/or growth pressure) for the volume elements. This enables the model to map the biological growth of the first tissue area (e.g., of the volume elements). As an alternative or in addition, the simulation of the biological growth may be based on standard values and/or a statistical model, which are selected as a function of a classification of the first tissue area. For this, the identification of the mapping of the first tissue area in the dataset may include a classification, where a tissue type (e.g., a tumor type and/or a type of the mass) is classified with the aid of the dataset. The classification may take place manually (e.g., with the aid of an annotation by the medical operating personnel) or automatically (e.g., through a comparison of image values and/or characteristics of the first tissue area from the dataset with a database, such as a dictionary (fingerprinting) having image values and/or characteristics of different tissue types). In this case, the volume elements of the model may each be assigned a default value and/or a static value from the statistical model as the growth parameter.

The simulation of the biological growth may be based on a temporal and/or spatial evolution of the volume elements of the model with the aid of the respective growth parameter. A spatial relative positioning of the volume elements in the first tissue area may be taken into account in the simulation of the biological growth (e.g., volume elements under pressure, such as inner volume elements) may grow more slowly than volume elements with no pressure (e.g., outer volume elements arranged in an edge area of the first tissue area).

The model may further simulate the change in the first tissue area, which is able to be created by the control of the therapy apparatus in accordance with the therapy plan between the at least one first point in time and the at least one second point in time. For example, the model may simulate the change in the first tissue area as a function of the therapy apparatus between the at least one first point in time and the at least one second point in time. For example, the model may simulate the change in the first tissue area (e.g., directly) by removal of the volume elements influenced by the therapy apparatus in accordance with the therapy plan between the at least one first point in time and the at least one second point in time. As an alternative or in addition, the model may simulate the volume elements influenced by the therapy apparatus in accordance with the therapy plan between the at least one first point in time and the at least one second point in time to be classified as lysed tissue (e.g., liquefied cell mass). The model may further assign to the removed volume elements and/or the volume elements classified as lysed tissue changed growth parameters for the simulation of the biological growth. For example, the model may simulate a shrinkage and/or no growth for the volume elements classified as removed and/or as lysed tissue.

In one embodiment, the model may simulate the biological growth and the change in the first tissue area as evolution (e.g., common evolution) of the volume elements influenced and uninfluenced by the therapy apparatus between the at least one first point in time and the at least one second point in time. Further, the model may simulate an interaction between the volume elements influenced and uninfluenced by the therapy apparatus (e.g., a pressure effect), between the at least one first point in time and the at least one second point in time. For example, the volume elements classified as lysed tissue may slow down the biological growth of the volume elements bordering on the volume elements, uninfluenced by the therapy apparatus.

The model may further take into account the vascularization in the first tissue area in the simulation of the biological growth and the change in the first tissue area between the at least one first point in time and the at least one second point in time. For example, the model may adjust the simulation of the biological growth of volume elements of the first tissue area as a function of the vascularization of the first tissue area (e.g., speed the simulation up for a dense vascularization).

The model may further map the first state, through the simulation of the biological growth and the change in the first tissue area between the at least one first point in time and the at least one second point in time, to the second state. As an alternative or in addition, the model may map the second state through an inverse simulation of the biological growth and the change in the first tissue area between the at least one first point in time and the at least one second point in time to the first state. The inverse simulation of the biological growth of the first tissue area may include a simulated shrinkage of the first tissue area (e.g., of volume elements uninfluenced by the therapy apparatus), starting from the second state, between the at least one first point in time and the at least one second point in time. The inverse simulation of the change in the first tissue area may further include an occurrence and/or a growth of volume elements influenced by the therapy apparatus in the first tissue area, starting from the second state, with the volume elements being classified in accordance with the therapy plan as removed volume element and/or lysed tissue.

The model may provide the therapy plan as output data. In this case, the therapy plan may predetermine the at least one parameter for control of the therapy apparatus such that the first tissue area at the at least one second point in time would fulfill the second state, for the change in the first tissue area able to be created by the therapy apparatus in accordance with the therapy plan.

The provision of the therapy plan may, for example, include storage on a computer-readable memory medium and/or display on the display unit and/or transmission to a therapy apparatus and/or a provision unit.

The method of one or more of the present embodiments may make possible an improved (e.g., results-oriented) planning of a treatment of a mass. In one embodiment, based on the dataset and the second state having the therapy plan, the at least one parameter for control of the therapy apparatus may be provided. For example, using the method, a therapy plan for a treatment (e.g., palliative treatment) of, for example, a beneficial or malignant mass (e.g., a tumor) and/or a plastic surgery (e.g., for shaping the first tissue area) may be provided.

In a further embodiment of the method, the dataset may have at least one first tissue parameter of the first tissue area and/or of a further tissue area, with the further tissue area bordering on the first tissue area.

The first tissue parameter may have spatially (e.g., two-dimensional or three-dimensional) and/or temporally-resolved information about the first tissue area and/or about the further tissue area. For example, the first tissue parameter may describe a deformability and/or elasticity and/or porosity and/or composition and/or functional information (e.g., a degree of bleeding through and/or metabolic activity) of the first tissue area and/or the further tissue area. In one embodiment, the first tissue parameter may be determined by an analysis of the dataset (e.g., of the first medical image data, such as of the image values and/or the time intensity curves of the image points). As an alternative or in addition, the first tissue parameter may be acquired and/or provided by a different examination modality from the dataset (e.g., the first medical image data, such as by a biopsy and/or a histology and/or tumor type determination of the first and/or the further tissue area).

The further tissue area may include a further anatomical region of the examination object (e.g., a tissue and/or an organ, such as a hollow organ) and/or a vessel section (e.g., an artery and/or vein). In one embodiment, the further tissue area may border on the first tissue area at least partly (e.g., at the tissue border of first tissue area). In this case, the first and the further tissue area may be mechanically coupled in the region of the border (e.g., movement-coupled, such as by connective tissue) or be mechanically decoupled (e.g., movement-decoupled).

In one embodiment, the input data of the model may further be based on the first tissue parameter.

The embodiment may make possible a more precise simulation of the biological growth and/or the change in the first tissue area between the at least one first point in time and the at least second point in time by the model.

In a further embodiment of the method, the model may simulate a mechanical interaction between the first tissue area and the further tissue area.

In one embodiment, the model may simulate an exertion (e.g., mutual exertion) of force and/or pressure effect and/or shear force and/or shear tension and/or compressive force and/or adhesion between the first tissue area and the further tissue area (e.g., in the region of the border), as the mechanical interaction between the first and the further tissue area. The model may capture a mechanical interaction that exists at the at least one first point in time, with the aid of the dataset (e.g., with the aid of the first tissue parameter). The model may further simulate a mechanical interaction arising between the at least one first point in time and the at least one second point in time between the first tissue area and the further tissue area as part of the simulation of the biological growth and the change in the first tissue area. The further tissue area may have an inelastic structure (e.g., a bone structure) that cannot be displaced by the first tissue area. The mechanical interaction may, for example, slow down the biological growth of the first tissue area at least in some areas. Further, the mechanical interaction may influence (e.g., slow down or speed up) the change able to be created by the therapy apparatus (e.g., a reduction in the spatial extent) of the first tissue area. The model may further simulate a narrowing or squashing of a vessel section and/or hollow organ in the first tissue area and/or the further tissue area through the mechanical interaction. In one embodiment, the model may take account of the mechanical interaction between the first tissue area and the further tissue area in the simulation of the biological growth and the change in the first tissue area (e.g., as a boundary condition). In this case, the model may determine the therapy plan such that the mechanical interaction between the first tissue area and the further tissue area may be minimized at the at least one second point in time.

The model may also capture a vascularization in the further tissue area with the aid of the dataset (e.g., with the aid of the first tissue parameter). In this case, the vascularization may describe a density of blood vessels and/or lymphatic transport paths in the further tissue area. The model may further take account of the vascularization in the further tissue area during the simulation of the biological growth and the change in the first tissue area between the at least one first point in time and the at least one second point in time. For example, the model may adapt the simulation of the biological growth of volume elements of the first tissue area that adjoin the further tissue area, depending on the vascularization of the further tissue area (e.g., speed the simulation up for a dense vascularization).

The embodiment may make possible a more precise simulation by the model of the biological growth and/or the change in the first tissue area between the at least one first point in time and the at least second point in time.

In a further embodiment of the method, the identification of the mapping of the first tissue area may include an identification of a mapping of at least one critical structure and/or of an edge area of the first tissue area in the dataset. In this case, the model may leave out the at least one critical structure and/or the edge area in the simulation of the change in the first tissue area.

The identification of the mapping of the at least one critical structure (e.g., of a number of critical structures) of the first tissue area may include a segmentation and/or highlighting and/or classification and/or marking (e.g., annotation) of image points and/or an image area having a number of image points in the dataset, with the image points and/or with the imaging area mapping the critical structure. In this case, a mapping of at least one anatomical object (e.g., of a vessel section, such as of an artery and/or vein) and/or of an organ (e.g., of a hollow organ, such as a bile duct) and/or of at least one anatomical region may be identified as the mapping of the at least one critical structure of the first tissue area in the dataset, which, with an action by the therapy apparatus, may represent a potential danger for the examination object (e.g., a risk of bleeding). The at least one anatomical object and/or the at least one anatomical region may be arranged at least partly (e.g., completely) in the first tissue area. In this case, the mapping of the at least one critical structure may be identified, for example, with the aid of image values of the image points of the dataset (e.g., of the first medical image data) and/or with the aid of an anatomy atlas and/or with the aid of patient information about the examination object (e.g., anamnesis data). Further, the mapping of the at least one critical structure may be identified by a third input of the medical operating personnel using the input unit (e.g., annotated in the graphical display of the dataset). The identification of the mapping of the at least one critical structure of the first tissue area in the dataset may take place manually, semi-automatically, or fully-automatically. The identification of the mapping of the at least one critical structure in the dataset may further be based on a risk assessment (e.g., computer-implemented; a blood flow simulation and/or a simulation of a tissue perfusion and/or a simulation of a dissolved fluid accumulation) of anatomical objects and/or anatomical regions of the first tissue area. The identification of the mapping of the at least one critical structure in the dataset may also be based on the first tissue parameter.

The edge area may include a tissue area (e.g., a tissue layer) of the first tissue area that is arranged along the tissue border of the first tissue area within a predetermined spatial depth with regard to the tissue border. The identification of the mapping of the edge region may include an identification of a mapping of the tissue border of the first tissue area. In this case, the identification of the mapping of the edge area (e.g., of the mapping of the tissue border) may take place in a similar way to the identification of the mapping of the first tissue area. The spatial depth with regard to the tissue border may further be predetermined as constant or spatially variable (e.g., as a function of the first tissue parameters).

In one embodiment, the model may leave out the at least one critical structure and/or the edge area in the simulation of change in the first tissue area. The leaving out of the at least one critical structure and/or of the edge area may provide, in this case, that in the spatial region of the at least one critical structure and/or the edge area, no input (e.g., direct input) of energy by the therapy apparatus is simulated.

If the result of the simulation of the biological growth and the change in the first tissue area is that the second state may only be achieved insufficiently precisely by the change in the first tissue area able to be created by the therapy apparatus, the model may further be embodied to determine the therapy plan as additionally having a workflow instruction for treatment and/or therapy of the first tissue area (e.g., of the at least one critical structure). The workflow instruction may, for example, have information about endovascular setting of a stent in the at least one critical structure.

The embodiment may minimize a risk of scattering intact cells (e.g., tumor cells) from the first tissue area and/or a risk of bleeding in a treatment of the first tissue area in accordance with the therapy plan.

In a further embodiment of the method, the second state may define a value and/or a range of values for the spatial extent and/or the second tissue parameter of the at least one part of the first tissue area for the at least one second point in time and/or a period of time including a number of second points in time and/or up to a temporal maximum value.

In this case, the period of time that includes the number of second points in time may describe a validity span (e.g., a contiguous validity span), within which the spatial extent and/or the second tissue parameter of the at least one part of the first tissue area fulfills the second state (e.g., the value and/or the range of values). As an alternative or in addition, through the definition of the value and/or of the range of values for the spatial extent and/or the second tissue parameter of the at least one part of the first tissue area for the at least one second point in time, it may be provided that the spatial extent and/or the second tissue parameter fulfill the second state at least at the at least one second point in time. As an alternative or in addition, the second state may define the value and/or the second tissue parameter of the at least one part of the first tissue area up to a temporal maximum value, with the temporal maximum value lying at or after the at least one second point in time.

The at least one second point in time and/or the period of time and/or the temporal maximum value may be predetermined by a fourth input of the medical operating personnel using an input unit. As an alternative or in addition, the model may be configured to determine the period of time and/or the temporal maximum value as part of the therapy plan. For this, the model may simulate the biological growth and the change in the first tissue area beyond the at least one second point in time. The at least one second point in time and/or the period of time and/or the temporal maximum value may describe a temporal validity range, within which the first tissue area fulfils the requirements of the of the second state.

In one embodiment, the at least one second point in time and/or the period of time and/or the temporal maximum value may be predetermined and/or determined as the point in time of further treatment of the first tissue area (e.g., by the same therapy apparatus). The at least one second point in time and/or the period of time and/or the temporal maximum value may further be predetermined and/or determined as a function of patient information (e.g., an age of the examination object).

In one embodiment, the second state may define the value (e.g., a minimum value or a maximum value) and/or the range of values (e.g., a validity range between a minimum value and a maximum value) for the second tissue parameter of the at least one part of the first tissue area (e.g., of the entire first tissue area) for the at least one second point in time and/or the period of time including a number of second points in time and/or up to the temporal maximum value. As an alternative or in addition, the second state may define the value (e.g., a maximum value) and/or the range of values (e.g., a validity range between a minimum value and a maximum value) for the spatial extent of the at least one part of the first tissue area (e.g., of the entire first tissue area) for the at least one second point in time and/or the period of time including a number of second points in time and/or the temporal maximum value.

When a period of time including a number of second points in time and/or a temporal maximum value that lies after the at least one second point in time is predetermined, the inverse simulation of the change in the first tissue area may include an occurrence and/or a growth of volume elements influenced by the therapy apparatus in the first tissue area starting from the second state. The occurrence and/or the growth of volume elements influenced by the therapy apparatus in the inverse simulation is simulated inversely as from the at least one second point in time (e.g., as from the earliest of the number of second points in time). This enables it to be provided that the volume elements of the first tissue area able to be influenced by the therapy apparatus in accordance with the therapy plan will be removed completely from the first tissue area and/or shrunk after the at least one second point in time (e.g., after the earliest of the number of second points in time).

Using the embodiment, the therapy plan for an improved (e.g., long-term) adherence to the second state by the first tissue area may be provided.

In a further embodiment of the method, the first tissue area may border on a further tissue area in at least one region. In this case, the second state may define the value and/or the range of values for the spatial extent and/or the second tissue parameter at least for the border.

The border in this case may describe a spatial region along the border surface of the first tissue area, in which the first tissue area and the further tissue area in the first state (e.g., at the at least one first point in time) and/or at the at least one second point in time, border on each other. As an alternative or in addition, the border may include an interstitial region between the first tissue area and the further tissue area in the first state and/or at the at least one second point in time. The simulated change of the first tissue area between the at least one first point in time and the at least one second point in time may result in a change in the border between the first tissue area and the further tissue area. For example, the change in the first tissue area may create and/or change the interstitial region between the first and the further tissue area.

In one embodiment, the second state may define the value and/or the range of values for the spatial extent and/or the second tissue parameter at least for the border. For example, the maximum value for the spatial extent of the at least one part of the first tissue area may be predetermined directly or indirectly (e.g., as a minimum value of a spatial extent of the further tissue area) that borders on the first tissue area, and/or as a minimum value of a spatial extent of the interstitial region between the first and the further tissue area.

In one embodiment, this enables improved taking into account of the further tissue area in the determination of the therapy plan to be made possible.

In a further form of embodiment of the method, by control of the therapy apparatus in accordance with the therapy plan, at least one lesion may be able to be created in the first tissue area. In this case, the model may simulate a decrease of the spatial extent of the first tissue area and/or a change in the second tissue parameter as the change between the at least one first point in time and the at least one second point in time. Further, the simulation of the simulation may include the change in transporting away (e.g., physiological) of lysed tissue from the at least one lesion.

In one embodiment, through the control of the therapy apparatus in accordance with the therapy plan, a lysis (e.g., spatially restricted lysis) of cells in the first tissue area between the at least one first point in time and the at least one second point in time may be able to be created, for example, by the, for example, temporally and/or spatially restricted and/or pulsed and/or continuous and/or focused input of energy into the first tissue area in accordance with the therapy plan. In this case, the input of energy may take place, for example, by mechanical (e.g., acoustic) and/or electromagnetic waves (e.g., ultrasound and/or microwaves and/or laser light). In one embodiment, the change may be able to be created without surgical access (e.g., open surgical access) in the first tissue area by the control of the therapy apparatus in accordance with the therapy plan. For this, the therapy apparatus may be configured to couple the mechanical and/or electromagnetic waves into the examination object (e.g., the first tissue area, such as by a coupling medium). A number of lesions may further be able to be created sequentially or simultaneously in the first tissue area through the control of the therapy apparatus in accordance with the therapy plan. The therapy apparatus may further be able to be arranged at least partly within the examination object (e.g., within a hollow organ of the examination object). In one embodiment, the therapy plan (e.g., the at least one parameter) may have information for control (e.g., regulation) of the energy input by the therapy apparatus between the at least one first point in time and the at least one second point in time. The at least one parameter of the therapy plan may, for example, predetermine an amplitude and/or pulse duration and/or current strength and/or voltage and/or temperature and/or frequency and/or phase and/or wavelength of the mechanical and/or electromagnetic waves (e.g., spatially and/or temporally resolved). Further, the at least one parameter of the therapy plan may predetermine a focus position and/or focus form and/or focus extent and/or focus direction of the energy input (e.g., of the mechanical and/or electromagnetic waves) and/or a positioning of the therapy apparatus (e.g., spatially and/or temporally resolved).

For example, the lysis of the cells may be able to be created in a, for example, contiguous spatial area or at a number of spatial areas (e.g., separated from one another by tissue, such as at points) within the first tissue area, through the control of the therapy apparatus in accordance with the therapy plan. The at least one spatial area (e.g., the number of spatial areas) including the lysed tissue (e.g., the lysed cells) may in this case form the at least one lesion (e.g., a number of lesions) in the first tissue area. The at least one lesion may thus be a cavity filled with lysed tissue within the first tissue area.

A physiological transporting away of the lysed tissue from the at least one lesion (e.g., the first tissue area, such as by a resorption) enables the lesion (e.g., the cavity) to collapse. Through this, the spatial extent (e.g., volume) of the first tissue area may reduce. As an alternative or in addition, the result may be a change in the second tissue parameter (e.g., the deformability and/or the elasticity and/or the porosity).

In one embodiment, the model may simulate the change in the first tissue area (e.g., the creation of at least one lesion by the energy input of the therapy apparatus in accordance with the therapy plan and the transporting away of the lysed tissue from the at least one lesion), between the at least one first point in time and the at least one second point in time. For example, the model may simulate the reduction of the spatial extent of the first tissue area (e.g., the collapse of the at least one lesion) and/or the change in the second tissue parameter as the change between the at least one first point in time and the at least one second point in time. In this case, the simulation of the change may include a simulation of the transporting away of lysed tissue (e.g., a shrinkage and/or a removal of volume elements classified as lysed tissue) from the at least one lesion. For example, the model may simulate the physiological transporting away (e.g., the resorption) of the lysed tissue from the at least one lesion. As an alternative, the model may simulate a drainage of the lysed tissue from the at least one lesion.

The model may further simulate the collapse of the at least one lesion, which may be caused by the simulated biological growth of the volume elements of the first tissue area uninfluenced by the therapy apparatus and/or by an interaction between the volume elements of the first tissue area and/or a mechanical interaction with the border with further tissue. In combination, the simulated biological growth and the change in the first tissue area between the at least one first point in time and the at least one second point in time may lead to a simulated reduction in the spatial extent of the first tissue area.

The model may simulate the processes described here (e.g., the simulated change and the simulated biological growth of the first tissue area), starting from the second state (e.g., inversely).

The embodiment may make possible an especially precise determination of the therapy plan.

In a further embodiment of the method, the therapy apparatus may have an ultrasound unit. In this case, therapeutic ultrasound may be able to be emitted by the ultrasound unit during the control of the therapy apparatus in accordance with the therapy plan into the first tissue area. Further, the at least one parameter may predetermine a frequency, a bandwidth, an amplitude, a phase, a pulse duration, a focus position, a beam alignment, a beam shape, and/or a duty cycle of the therapeutic ultrasound such that the at least one lesion in the tissue area is able to be created in the first tissue by the therapeutic ultrasound.

A totality of the ultrasound coupled by the ultrasound unit into the examination object (e.g., a coupling-in ultrasound field) will be referred to below as therapeutic ultrasound.

In one embodiment, the ultrasound unit may have at least one ultrasound transducer that is configured for emitting the therapeutic ultrasound (e.g., the ultrasound field) in accordance with the therapy plan. In this case, the ultrasound field may describe a field of a spatial propagation of sound waves. In the control of the therapy apparatus in accordance with the therapy plan, the therapeutic ultrasound (e.g., the ultrasound field) may be able to be emitted by the ultrasound unit within an opening angle essentially in a first main direction (e.g., in a fan shape and/or a cone shape and/or focused on the focus position). Further, the therapeutic ultrasound may be able to be emitted by the ultrasound unit continuously or pulsed during the control of the therapy apparatus in accordance with the therapy plan.

The at least one parameter (e.g., the number of parameters) of the therapy plan may predetermine the frequency, the bandwidth, the amplitude, the phase, the pulse duration, the focus position, the beam alignment, the beam shaping, and/or the duty cycle of the therapeutic ultrasound (e.g., of the ultrasound field), such that the at least one lesion (e.g., the spatially restricted lysis of tissue) is able to be created in the first tissue area. In one embodiment, the dataset (e.g., the first tissue parameter) may, for example, have spatially resolved information about a threshold value (e.g., a destruction threshold) for an ultrasound-induced lysis in the first tissue area. In this case, the threshold value may predetermine a lower ultrasound intensity limit, at which the lysis of tissue in the first tissue area is able to be created (e.g., locally or regionally) when the threshold value is reached or exceeded. In one embodiment, the at least one lesion may be created by warming (e.g., heating up by highly intensive focused ultrasound (HIFU)) and/or by mechanical destruction (e.g., a histotripsy as subtype of HIFU) of the tissue to be lysed. For example, the at least one lesion may be able to be created by the therapeutic ultrasound able to be emitted by the ultrasound unit into the first tissue area during the control the therapy apparatus in accordance with the therapy plan.

In one embodiment, the model may simulate the change in the first tissue area (e.g., the creation of the at least one lesion by the therapeutic ultrasound able to be emitted by the ultrasound unit in accordance with the therapy plan and the transporting away of the lysed tissue from the at least one lesion) between the at least one first point in time and the at least one second point in time.

This enables a precise simulation of the change in the first tissue area able to be created by the therapeutic ultrasound to be made possible using the model.

In a further embodiment of the method, the determination of the therapy plan may include the acts a.1) to b.2). In act a.1), a first simulated state of the first tissue area for the at least one first point in time may be simulated by inverse simulation of the biological growth and the change in the first tissue area between the at least one first point in time and the at least one second point in time starting from a second initial state. In act a.2), the first simulated state may be adjusted to the first state by adjusting the at least one parameter of the therapy plan. The adjusted first simulated state may further be provided as the first initial state. Further, in act b), a second simulated state of the first tissue area for the at least one second point in time may be determined by simulation of the biological growth and the change in the first tissue area between the at least one first point in time and the at least one second point in time staring from a first initial state. Further, in act b.2), the second simulated state may be adjusted to the second state by adapting the at least one parameter of the therapy plan. Further, the adjusted second simulated state may be predetermined as the second initial state. In this case, the acts a.1) to b.2) may be executed repeatedly until the occurrence of an abort condition. When the acts a.1) to b.2) are first executed, execution begins with act a.1). Further, the second state may be predetermined as the second initial state. As an alternative, when the acts a.1) to b.2) are first executed, execution may begin with act b.1). In this case, the first state may be predetermined as the first initial state. In both cases, a start value for the at least one parameter of the therapy plan may be received, geometrically predetermined, or initialized by a random seed generator.

The first simulated state of the first tissue area may have a virtual representation of the first tissue area at the at least one first point in time (e.g., a volume mesh model). The first simulated state may, for example, describe all characteristics of the first tissue area at the at least one first point in time, which are described for the at least one first point in time by the first state. Similarly to this, the second simulated state of the first tissue area may have a virtual representation of the first tissue area at the at least one second point in time (e.g., a volume mesh model). The second simulated state may further describe all characteristics of the first tissue area at the at least one second point in time, which are described for the at least one first point in time by the first state.

The model may determine the first simulated state by inverse simulation (e.g., temporal backward simulation) of the biological growth and the change in the first tissue area (e.g., of the volume elements of the first tissue area) starting from the second initial state between the at least one first and the at least one second point in time. The start value for the at least one parameter of the therapy plan may be received on first execution or initialized by the random seed generator. The receipt of the start value for the at least one parameter of the therapy plan may, for example, include an acquisition and/or readout of a computer-readable data memory and/or a receipt from a data memory unit (e.g., a database). Further, the start value may be predetermined by a fifth input of a medical operator using the input unit. The start value may further be provided based on an artificial intelligence.

As an alternative or in addition, the start value for the at least one parameter of the therapy plan may be predetermined geometrically (e.g., based on geometrical methods). For example, an ellipsoid (e.g., spherical) region within the at least one lesion (e.g., at a geometrical central point of the at least one lesion) may be predetermined as the start value. When the identification of the mapping of the first tissue area includes an identification of a mapping of at least one critical structure and/or of an edge area of the first tissue area in the dataset, then a spatial area (e.g., a volume) within the first tissue area without the at least one critical structure and/or the edge area may be predetermined as the start value for the at least one parameter of the therapy plan.

The adjustment of the first simulated state to the first state may be based on a comparison of the first simulated state with the first state. In this case, the comparison of the first simulated state with the first state of the first tissue area may include a comparison of the characteristics of the first tissue area described by the two states (e.g., the spatial extent of the first tissue area). Through comparison of the first simulated state with the first state of the first tissue area, a deviation between the first simulated state and the first state of the first tissue area may be determined. The at least one parameter of the therapy plan may further be adjusted iteratively depending on the deviation between the first simulated state and the first state of the first tissue area. In addition, the adjustment of the first simulated states to the first state may include an application of a geometrical transformation (e.g., a translation and/or rotation and/or scaling) to the first simulated state. The adjustment of the first simulated state to the first state may take place such that the deviation between the first simulated state and the first state of the first tissue area is minimized. Further, the adjustment of the first simulated state to the first state may include an optimization (e.g., minimization) of a cost value of a first cost function. The first cost function characterizes (e.g., quantifies) the deviation between the first simulated state and the first state of the first tissue area.

The model may further determine the second simulated state by simulation (e.g., temporal forwards simulation) of the biological growth and the change in the first tissue area (e.g., of the volume elements of the first tissue area) starting from the first initial state, between the at least one first point in time and the at least one second point in time. The adjustment of the second simulated state to the second state by the adjustment of the at least one parameter of the therapy plan may, for example, take place in a similar way to the adjustment of the first simulated state to the first state. In one embodiment, the adjustment of the second simulated state to the second state may take place such that a deviation between the second simulated state and the second state of the first tissue area is minimized. Further, the adjustment of the second simulated state to the second state may include an optimization (e.g., minimization) of a cost value of a second cost function. The second cost function characterizes (e.g., quantifies) the deviation between the second simulated state and the second state of the first tissue area.

In one embodiment, the acts a.1) to b.2) may be executed repeatedly until the abort condition occurs. In this case, the abort condition may include a comparison of the cost values of the first cost function and/or the second cost function with a predetermined threshold value. As an alternative or in addition, the abort condition may predetermine a maximum number of repetitions of the acts a.1) to b.2).

Further, the determination of the therapy plan may be carried out (e.g., in parallel) for a number of different start values (e.g., an ensemble of start values) of the at least one parameter of the therapy plan. In this case, after a predetermined number of repetitions of the acts a.1) to b.2), a convergence behavior of the parallel instances with regard to the first and the second is assessed. If a number of the parallel instances converge within the predetermined threshold values, there may be a downstream assessment of the treatment duration and/or treatment frequency in accordance with the treatment plans determined.

The embodiment may make possible a robust and precise determination of the therapy plan.

In a further embodiment of the method, the model may include a trained function. In this case, at least one parameter of the trained function may be adjusted by a comparison of a training therapy plan with a comparison therapy plan.

The trained function may be trained by a machine learning method. For example, the trained function may be a neural network (e.g., a convolutional neural network (CNN) or a network include a convolutional layer).

The trained function maps input data to output data. The output data may, for example, also depend on one or more parameters of the trained function. The parameter or the number of parameters of the trained function may be determined and/or adjusted by training. The determination and/or the adjustment of the parameter or the number of parameters of the trained function may, for example, be based on a pair consisting of training input data and associated training output data (e.g., comparison output data). The trained function for creation of training mapping data is applied to the training input data. For example, the determination and/or the adjustment may be based on a comparison of the training mapping data and the training output data (e.g., of the comparison output data). In general, a trainable function, a function with one or more parameters not yet adjusted, may be referred to as a trained function.

Other terms for trained function are trained mapping rule, mapping rule with trained parameters, function with trained parameters, algorithm based on artificial intelligence, and machine-learning algorithm. An example of a trained function is an artificial neural network, where the edge weights of the artificial neural network correspond to the parameters of the trained function. Instead of the term “neural network”, the term “neural net” may also be used. For example, a trained function may also be a deep artificial neural network, deep neural network. A further example of a trained function is a “support vector machine”; further, other machine learning algorithms are, for example, also able to be employed as the trained function.

The trained function may, for example, be trained by a back propagation. Training mapping data may be determined by application of the trained function to training input data. Hereafter, a deviation between the training mapping data and the training output data (e.g., the comparison output data) may be established by application of an error function to the training mapping data and the training output data (e.g., the comparison output data). Further, at least one parameter (e.g., a weighting) of the trained function (e.g., of the neural network) based on a gradient of the error function may be adjusted iteratively with regard to the at least one parameter of the trained function. This enables the deviation between the training mapping data and the training output data (e.g., the comparison output data) to be minimized during the training of the trained function.

In one embodiment the trained function (e.g., the neural network) has an input layer and an output layer. In this case, the input layer may be configured for receipt of input data. The output layer may further be configured for provision of mapping data. In this case, the input layer and/or the output layer may each include a number of channels (e.g., neurons).

The input data of the trained function may be based on the dataset (e.g., the first medical image data and/or the first tissue parameter) and the second state (e.g., the second tissue parameter). The trained function having the therapy plan may further provide the at least one parameter for control of the therapy apparatus as output data.

In one embodiment, the at least one parameter of the trained function may be adjusted by a comparison of a training therapy plan with a comparison therapy plan. For example, the trained function may be provided by an embodiment of the computer-implemented method for provision of a trained function that is described further on in the course of this document.

The embodiment may make possible an efficient provision of the therapy plan in terms of computing.

In a further embodiment of the method for provision of a therapy plan, the dataset may map an initial change in the first tissue area. In this case, the initial change in the first tissue area may have been created before the beginning of the method by the therapy apparatus in accordance with an initial therapy plan. The dataset may also have the initial therapy plan.

The dataset may map the initial change in the first tissue area (e.g., on a time-resolved basis) at a number of first points in time. The initial therapy plan may have all features and characteristics that have been described with regard to the therapy plan. For example, the initial therapy plan may have been provided by a form of embodiment of the proposed method, where an initial second state has been specified for the first tissue area. In this case, the dataset may map the initial change in the first tissue area before or up to reaching the initial second state.

In one embodiment, the dataset may also have the initial therapy plan and/or the initial second state. In this case, the input data of the model may also be based on the initial therapy plan. Provided the dataset has the initial second state, the second state may be specified based on the initial second state. For example, the second state may be specified as the initial second state.

In one embodiment, the model may use the initial therapy plan as the start value for the determination of the therapy plan. Further, the model may adapt the simulation of the biological growth and/or the change in the first tissue area based on the mapping of the initial change in the dataset.

The embodiment may make possible simulation of the biological growth and/or the change in the first tissue area between the at least one first point in time and the at least second point in time by the model adjusted to the initial change.

In a further embodiment of the method, the provision of the therapy plan may include a transmission of the at least one parameter to the therapy apparatus and/or a preparatory adjustment of at least one operating parameter and/or of at least one positioning parameter of the therapy apparatus in accordance with the therapy plan (e.g., without the control of the therapy apparatus).

The transmission of the at least one parameter to the therapy apparatus may take place in a data structure adjusted to the therapy apparatus and/or an adjusted data format and/or transmission protocol. The preparatory adjustment of the at least one operating parameter and/or of the at least one positioning parameter of the therapy apparatus may include a presetting and/or initialization of the at least one operating parameter and/or of the at least one positioning parameter to the at least one parameter of the therapy plan. For example, the provision of the therapy plan may include a translation of the therapy plan adjusted to the therapy apparatus including the at least one parameter into a protocol including the at least one operating parameter and/or the at least one positioning parameter of the therapy apparatus.

The embodiment may make possible an improved provision of the therapy plan.

In accordance with a further embodiment of the method, the provision of the therapy plan may include a control of the therapy apparatus in accordance with the therapy plan. The change between the at least one first point in time and the at least one second point in time in the first tissue area is created. In this case, the therapy apparatus may create the change in the first tissue area in accordance with the therapy plan (e.g., the at least one parameter) between the at least one first point in time and the at least one second point in time.

In a second aspect, the present embodiments relate to a computer-implemented method for provision of a trained function. In this case, a preacquired first training dataset is received, which maps an examination object including a first tissue area at at least one first point in time. A mapping of the first tissue area in the first training dataset is further identified. Further, a preacquired second training dataset is received, which maps the examination object including the first tissue area at at least one second point in time after the first point in time. Further, a further mapping of the first tissue area in the second training dataset is identified as a second training state. A comparison therapy plan for a therapy apparatus is further received. In this case, the comparison therapy plan has an instruction and/or a parameter for control of the therapy apparatus. Further, before the beginning of the method, between the at least one first point in time and the at least one second point in time, a change in the first tissue area by the therapy apparatus is created in accordance with the comparison therapy plan. Further, a training therapy plan for the therapy apparatus is determined by applying the trained function to input data. In this case, the input data is based on the first training dataset and the second training state. Further, the training therapy plan is provided as output data of the trained function. At least one parameter of the trained function is further adjusted by a comparison of the training therapy plan with the comparison therapy plan. Hereafter, the trained function is provided.

In this case, the acts of the method for provision of a trained function described may be carried out after one another and/or at least partly simultaneously.

The receipt of the first training dataset and/or the second training dataset may include an acquisition and/or readout of a computer-readable data memory and/or a receipt from a data memory unit (e.g., a database). The first training dataset may further be provided by a provision unit of a medical imaging device for recording the first training dataset. Further, the second training dataset may be provided by a provision unit of a medical imaging device for recording the second training dataset. In this case, the medical imaging unit for recording the second training dataset may be the same as or different from the medical imaging device for recording the first training dataset.

The first training dataset may, for example, have all the characteristics of the dataset that have been described with regard to the method for provision of a therapy plan and vice versa. The identification of the mapping of the first tissue area in the first training dataset may further take place similarly to the identification of the mapping of the first tissue area in the dataset, which has been described with regard to the method for provision of a therapy plan.

The second training dataset may have second medical image data that maps the examination object including the first tissue area spatially two-dimensionally and/or three-dimensionally resolved. In this case, the second training dataset (e.g., the second medical image data) may map the examination object including the first tissue area on a time-resolved basis at at least one second point in time (e.g., at a number of second points in time). The second training dataset (e.g., the second medical image data) may have a number of image points with image values that map the examination object including the first tissue area. Provided the second training dataset maps the examination object including the first tissue area on a time-resolved basis, the image points may have time intensity curves. In one embodiment, the second training dataset (e.g., the second medical image data) may be recorded by the medical imaging device for recording the second training dataset at the at least one second point in time (e.g., at the number of second points in time; may be preacquired). The second training dataset may further have metadata. The metadata may, for example, include information about a recording parameter and/or operating parameter of the medical imaging device for recording the second training dataset. In one embodiment, the second training dataset may be registered with the first training dataset.

The identification of the further mapping of the first tissue area in the second training dataset may take place similarly to the identification of the mapping of the first tissue area in the first training dataset. The second training state may be identified by characteristics (e.g., all characteristics) of the first tissue area that the second training dataset has for the identified further mapping of the first tissue area (e.g., an identified further image region). The second training state may, for example, have all features and characteristics that have been described with regard to the second state and vice versa.

The receipt of the comparison therapy plan may include an acquisition and/or readout of a computer-readable data memory and/or a receipt from a data memory unit (e.g., a database). The comparison therapy plan may further be provided by a provision unit of the therapy apparatus and/or by a further input of the medical operating personnel using an input unit.

The comparison therapy plan may, for example, have all characteristics of the therapy plan that have been described with regard to the method for provision of a therapy plan and vice versa. In this case, the comparison therapy plan may have the at least one parameter (e.g., an operating parameter and/or a positioning parameter) that was used before the beginning of the method between the at least one first point in time and the at least one second point in time for control of the therapy apparatus. Thus, the comparison therapy plan (e.g., the at least one parameter) may describe the control of the therapy apparatus between the at least one first point in time and the at least one second point in time. Further, before the beginning of the method, through a control of the therapy apparatus in accordance with the comparison therapy plan (e.g., the at least one parameter), the change of the first tissue area between the at least one first point in time and the at least one second point in time may have been created.

The second training dataset (e.g., the second medical image data) may map the first tissue area at the at least one second point in time (e.g., after the change by the therapy apparatus in accordance with the comparison therapy plan). Thus, the second training dataset may, for example, map all differences at the first tissue area that have been caused by the change between the at least one first point in time and the at least one second point in time.

The training therapy plan is further determined by application of the trained function to the input data. In this case, the input data is based on the first training dataset and the second training state. The training therapy plan is further provided as output data of the trained function. Further, through the comparison between the training therapy plan and the comparison therapy plan, the at least one parameter of the trained function is adjusted. The comparison between the training therapy plan and the comparison therapy plan may include a comparison of the at least one parameter of the training therapy plan with the at least one parameter of the comparison therapy plan. For example, the comparison between the training therapy plan and the comparison therapy plan may include a determination of a deviation between the training therapy plan and the comparison therapy plan (e.g., between the at least one parameter of the training therapy plan and the at least one parameter of the comparison therapy plan). In this case, the at least one parameter of the trained function may be adjusted such that the deviation between the training therapy plan and the comparison therapy plan is minimized. The adjustment of the at least one parameter of the trained function may, for example, include an optimization (e.g., minimization) of a cost value of a third cost function. The third cost function characterizes (e.g., quantifies) the deviation between the training therapy plan and the comparison therapy plan. For example, the adjustment of the at least one parameter of the trained function may include a regression of the cost value of the third cost function.

With the method, a trained function may be provided, which may be used in an embodiment of the method for provision of a therapy plan.

The provision of the trained function may, for example, include storage on a computer-readable memory medium and/or transmission to a provision unit.

In a third aspect, the present embodiments relate to a provision unit that is configured to carry out an embodiment of the method for provision of a therapy plan.

In one embodiment, the provision unit may include a computing unit, a memory unit, and/or an interface. The provision unit may be configured to carry out a form of embodiment of the method for provision of a therapy plan and its aspects, in that the interface, the processing unit, and/or the memory unit are configured to carry out the corresponding method acts. For example, the interface may be configured for receipt of the preacquired dataset and/or for provision of the therapy plan. The computing unit and/or the memory unit may further be configured for identification of the mapping of the first tissue area and/or for specification of the second state and/or for determination of the therapy plan.

The advantages of the provision unit essentially correspond to the advantages of the method for provision of a therapy plan. Features, advantages, or alternate forms of embodiment mentioned may likewise be transferred to other subject matter and vice versa.

In a fourth aspect, the present embodiments relate to a system having a proposed provision unit and a therapy apparatus. In this case, the therapy apparatus is configured to create the change in the first tissue area. The provision unit is further configured to control the therapy apparatus in such a way with the aid of the therapy plan that the change between the at least one first point in time and the at least one second point in time is created in the first tissue area.

The therapy apparatus may be configured to create the change in the first tissue area between the at least one first point in time and the at least one second point in time via a temporally and/or spatially restricted (e.g., pulsed and/or continuous and/or focused) energy input into the first tissue area in accordance with the therapy plan. In this case, the energy input may be configured, for example, by mechanical (e.g., acoustic) and/or electromagnetic waves (e.g., ultrasound and/or microwaves and/or laser light). In one embodiment, the therapy apparatus may be configured to create the change without surgical access (e.g., open surgical access) in the first tissue area. For this, the therapy apparatus may be configured to couple the mechanical and/or electromagnetic waves into the examination object (e.g., the first tissue area, by a coupling medium). The therapy apparatus may be further configured to be arranged at least partly within the examination object (e.g., within a hollow organ of the examination object).

The advantages of the system essentially correspond to the advantages of the method for provision of a therapy plan. Features, advantages, or alternate forms of embodiment mentioned may likewise be transferred to other subject matter and vice versa.

In a further embodiment of the system, the therapy apparatus may have an ultrasound unit. In this case, the ultrasound unit may be configured to send therapeutic ultrasound into the first tissue area. The therapeutic ultrasound may be configured to create at least one lesion in the first tissue area as the change.

A totality of the ultrasound coupled by the ultrasound unit into the examination object (e.g., the ultrasound field) will be referred to below as therapeutic ultrasound.

In one embodiment, the ultrasound unit may have at least one ultrasound transducer that is configured to emit the therapeutic ultrasound (e.g., of an ultrasound field) in accordance with the therapy plan. In this case, the ultrasound field may describe a field of a spatial propagation of sound waves. Further, the ultrasound unit may have a coupling unit (e.g., with a tank) that may be filled with a coupling medium (e.g., a fluid and/or a gel). The coupling unit may further have on its outer side a membrane (e.g., a deformable membrane) that is configured to adapt itself to a surface shape of the examination object (e.g., flexibly). The coupling unit may be configured to couple the at least one ultrasound transducer acoustically to a surface of the examination object. This enables the therapeutic ultrasound to be coupled through the surface of the examination object (e.g., the skin) into the first tissue area. The at least one ultrasound transducer may be configured to emit the therapeutic ultrasound (e.g., the ultrasound field) within an opening angle essentially in a first main direction (e.g., in a fan shape and/or in a cone shape). Further, the at least one ultrasound transducer may be configured to emit the therapeutic ultrasound (e.g., the ultrasound field) having a predetermined frequency and/or bandwidth and/or amplitude and/or phase and/or pulse duration and/or predetermined duty cycle predetermined by the therapy plan (e.g., the at least one parameter of the therapy plan, pulsed or continuously).

The ultrasound unit may further have a number of ultrasound transducers that are arranged in a 2D or 3D array (e.g., a matrix array). In this case, the ultrasound unit (e.g., the number of ultrasound transducers) may be configured for focused emission of the therapeutic ultrasound (e.g., of the ultrasound field). In one embodiment, the ultrasound unit may be configured to adapt a spatial position of the focusing of the ultrasound and/or a beam alignment and/or beam shaping of the ultrasound field depending on the therapy plan.

The therapeutic ultrasound may be configured to create the at least one lesion (e.g., the spatially restricted lysis of tissue) in the first tissue area. In one embodiment, the dataset (e.g., the first tissue parameter) may have information (e.g., spatially resolved) about a threshold value (e.g., a destruction threshold) for an ultrasound-induced lysis in the first tissue area. In this case, the threshold may predetermine an ultrasound intensity limit at which, when reached or exceeded, the lysis of tissue in the first area may be created (e.g., locally or regionally). In one embodiment, the therapeutic ultrasound may be configured to create the at least one lesion by warming (e.g., heating up by highly intensive focused ultrasound (HIFU), and/or by mechanical destruction, such as a histotripsy as subtype of the HIFU) of the tissue to be lysed.

In one embodiment, the model may simulate the change of the first tissue area (e.g., the creation of the at least one lesion by the therapeutic ultrasound emitted by the ultrasound unit in accordance with the therapy plan and the transporting away of the lysed tissue from the at least one lesion) between the at least one first point in time and the at least one second point in time. This enables the therapy plan to be determined especially precisely. An improved control of the therapy apparatus in accordance with the therapy plan may further be made possible.

In a further embodiment of the system, the system may further have a medical imaging device. In this case, the medical imaging device may be configured to accept and/or provide the dataset.

The medical imaging device may, for example, be configured as a magnetic resonance tomography installation (MRT) and/or computed tomography installation (CT) and/or medical x-ray device and/or positron emission tomography installation (PET) and/or ultrasound device.

The present embodiments may further relate to a training unit that is configured to carry out a form of embodiment of the proposed computer-implemented method for provision of a trained function.

In one embodiment, the training unit may include a training computing unit, a training memory unit, and/or a training interface. The training unit may be configured to carry out an embodiment of the method for provision of a trained function and its aspects, in that the training interface, the training computing unit, and/or the training memory unit are configured to carry out the corresponding method acts. For example, the training interface may be configured for receipt of the preacquired first training dataset and/or of the preacquired second training dataset and/or for receipt of the comparison therapy plan and/or for provision of the trained function. The computing unit and/or the memory unit may be further configured for identification of the mapping of the first tissue area and/or for identification of the further mapping of the first tissue area and/or for determination of the training therapy plan and/or for adjustment of the at least one parameter of the trained function.

The advantages of the training unit essentially correspond to the advantages of the method for provision of a therapy plan. Features, advantages, or alternate forms of embodiment mentioned may likewise be transferred to other subject matter and vice versa.

In a fifth aspect, the present embodiments relate to a computer program product with a computer program that is able to be loaded directly into a memory of a provision unit, with program sections for carrying out all acts of the method for provision of a therapy plan when the program sections are executed by the provision unit. Alternatively or additionally, the computer program is able to be loaded directly into a training memory of a training unit, with program sections for carrying out all acts of the method for provision of a trained function and/or of one of its aspects when the program sections are executed by the training unit.

The present embodiments may further relate to a computer-readable memory medium, on which program sections able to be read and executed by a provision unit are stored, for carrying out all acts of the method for provision of a therapy plan when the program sections are executed by the provision unit. Alternatively or additionally, program sections stored on the computer-readable memory medium are able to be read and executed by a training unit for carrying out all acts of the method for provision of a trained function and/or one of its aspects when the program sections are executed by the training unit.

The present embodiments may further relate to a computer program or computer-readable memory medium, including a trained function provided by an embodiment of the method for provision of a trained function or one of its aspects.

A largely software-based realization has the advantage that even provision units and/or training units already used previously may be upgraded in a simple way by a software update in order to work according to the present embodiments. Such a computer program product, as well as the computer program, may, where necessary, include additional elements such as, for example, documentation and/or additional components, as well as hardware components, such as, for example, hardware keys (e.g., dongles, etc.) for using the software.

### BRIEF DESCRIPTION OF THE DRAWINGS

Exemplary embodiments of the invention are shown in the drawings and will be described in greater detail below. In different figures, the same reference characters are used for the same features. In the drawings:

FIG. 1 shows a schematic diagram of an embodiment of a method for provision of a therapy plan;

FIG. 2 shows a schematic diagram of one embodiment of an inverse simulation of biological growth and a change in a first tissue area,

FIGS. 3 to 5 show schematic diagrams of further embodiment of the method for provision of a therapy plan;

FIG. 6 shows a schematic diagram of an embodiment of a method for provision of a trained function;

FIG. 7 shows a schematic diagram of one embodiment of a system;

FIG. 8 shows a schematic diagram of one embodiment of a provision unit; and

FIG. 9 shows a schematic diagram of one embodiment of a proposed training unit.

### DETAILED DESCRIPTION

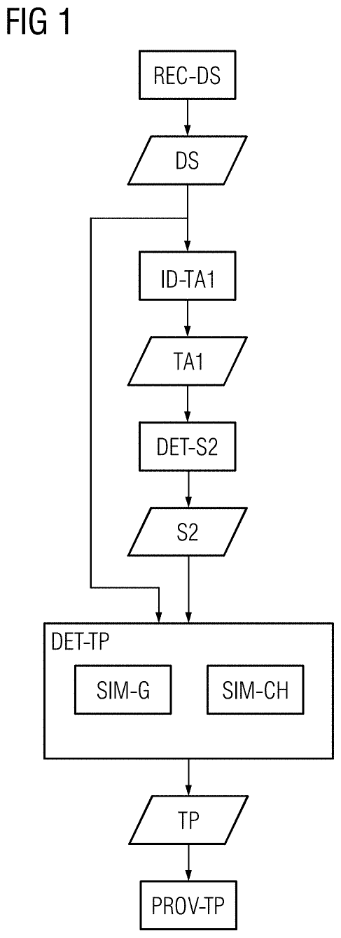

Shown schematically in FIG. 1 is an embodiment of a method (e.g., a computer-implemented method) for provision PROV-TP of a therapy plan TP. In a first act, a preacquired dataset DS may be received REC-DS. In this case, the dataset DS may map an examination object including a first tissue area TA1 at at least one first point in time. Further, in a second act, a mapping of the first tissue area TA1 may be identified ID-TA1 in the dataset DS as a first state. In a third act, a second state S2 may be specified DET-S2 for the first tissue area TA1. In this case, the second state S2 may define a spatial extent (e.g., a maximum) and/or a second tissue parameter of at least a part of the first tissue area TA1 at at least one second point in time after the at least one first point in time. In a fourth act, a therapy plan TP for a therapy apparatus may be determined by application of a model to input data DET-TP. In this case, the input data may be based on the dataset DS and the second state S2. The therapy plan TP may further have at least one parameter for control of the therapy apparatus. Further, a change in the first tissue area TA1 between the at least one first point in time and the at least one second point in time may be able to be created through the control of the therapy apparatus in accordance with the therapy plan. The model may further simulate a biological growth SIM-G and the change SIM-CH in the first tissue area between the at least one first point in time and the at least one second point in time. In this case, the model may map the first state to the second state S2 or the second state S2 to the first state. In a fifth act, the therapy plan TP may be provided PROV-TP as output data of the model.

In one embodiment, the identification ID-TA1 of the mapping of the first tissue area TA1 may include an identification of a mapping of at least one critical structure and/or of an edge area of the first tissue area TA1 in the dataset DS. In this case, the model may leave out the at least one critical structure and/or the edge area in the simulation of the change in the first tissue area TA1.

The second state S2 may further define a value and/or a range of values for the spatial extent and/or the second tissue parameter of the at least one part of the first tissue area TA1 for the at least one second point in time and/or a period of time including a number of second points in time and/or up to a temporal maximum value.

The dataset DS may further map an initial change in the first tissue area TA1 (e.g., on a time-resolved basis) at a number of first points in time. In this case, the initial change in the first tissue area TA1 may have been created before the beginning of the method by the therapy apparatus in accordance with an initial therapy plan. The dataset may additionally have the initial therapy plan.

In one embodiment, the provision PROV-TP of the therapy plan TP may include a transmission of the at least one parameter to the therapy apparatus and/or a preparatory adjustment of at least one operating parameter and/or at least one positioning parameter to the therapy apparatus in accordance with the therapy plan TP (e.g., without the control of the therapy apparatus).

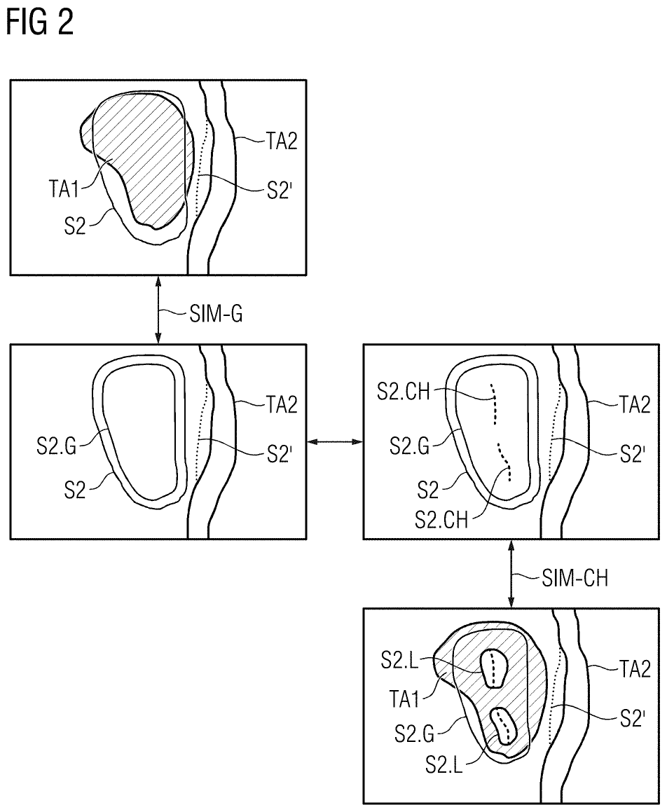

Shown schematically in FIG. 2 is a form of embodiment of a simulation (e.g., an inverse simulation) of the biological growth SIM-G and the change SIM-G in the first tissue area TA1. The model may determine the therapy plan TP through inverse simulation (e.g., temporal backward simulation) of the biological growth SIM-G and the change SIM-CH in the first tissue area TA starting from the second state S2 between the at least one first point in time and the at least one second point in time. In this case, the model may map the second state S2 to the first state. As an alternative or in addition, the model may determine the therapy plan TP through simulation (e.g., temporal forwards simulation) of the biological growth SIM-G and the change SIM-CH of the first tissue area TA starting from the first state between the at least one first point in time and the at least one second point in time. In this case, the model may map the first state to the second state S2.

Described below by way of example is the inverse simulation of the biological growth SIM-G and the change SIM-CH in the first tissue area TA starting from the second state S2. The first tissue area TA1 may border on a further tissue area TA2 in at least one area. The dataset may further have a mapping of the first tissue area TA1 and of the further tissue area TA2. Further, the second state S2 may define the value (e.g., the maximum value) and/or the range of values for the spatial extent of the first tissue area and/or the second tissue parameter at least for the border. For example, the maximum value for the spatial extent of the at least one part of the first tissue area TA1 may be predetermined directly or indirectly (e.g., as minimum value S2′ of a spatial extent of the further tissue area TA). Account may also be taken here of an interstitial area between the first tissue area TA1 and the further tissue area TA2.

The inverse simulation of the biological growth SIM-G of the first tissue area TA1 may include a simulated shrinkage of the first tissue area TA (e.g., of volume elements uninfluenced by the therapy apparatus) starting from the second state S2, between the at least one first point in time and the at least one second point in time. In FIG. 2, the result of the inverse simulation of the biological growth SIM-G (e.g., the result of the simulated shrinkage) of the first tissue area TA1 is illustrated by the simulated state S2.G.

The control of the therapy apparatus in accordance with the therapy plan TP enables at least one lesion S2.L to be able to be created in the first tissue area TA1. In this case, the model may simulate SIM-CH a reduction of the spatial extent of the first tissue area TA1 and/or a change in the second tissue parameter as the change between the at least one first point in time and the at least one second point in time. For this, the inverse simulation of the change SIM-CH of the first tissue area TA1 may include an occurrence (e.g., an appending) of volume elements S2.CH uninfluenced by the therapy apparatus in the first tissue area TA1 starting from the second state S2 or starting from the result S2.G of the inverse simulation of the biological growth SIM-G. Further, the inverse simulation of the change SIM-CH may include an inverse simulation of a, for example, physiological transporting away from lysed tissue from the at least one lesion S2.L. The at least one lesion S2.L may arise as a result of the inverse simulation of the change SIM-CH of the first tissue area TA1 from the volume elements influenced by the therapy apparatus S2.CH.

The therapy apparatus may further have an ultrasound unit. In this case, the therapeutic ultrasound may be able to be emitted into the first tissue area TA1 by the ultrasound unit during the control of the therapy apparatus in accordance with the therapy plan TP. The at least one parameter may further predetermine a frequency, a bandwidth, an amplitude, a phase, a pulse duration, a focus position, a beam alignment, a beam shaping, and/or a duty cycle of the therapeutic ultrasound such that the at least one lesion S2.L in the first tissue area TA.1 is able to be created by the therapeutic ultrasound.

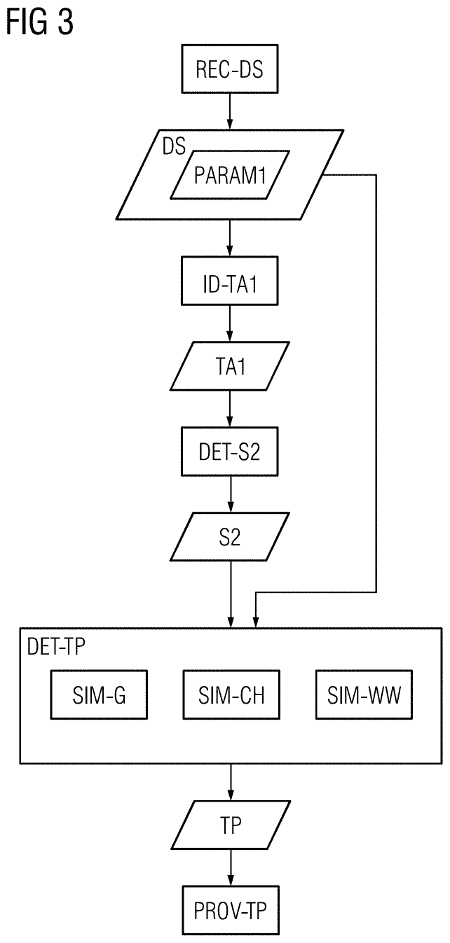

FIG. 3 shows a schematic diagram of a further embodiment of the method for provision PROV-TP of a therapy plan TP. In this embodiment, the dataset DS may have at least one first tissue parameter PARAM1 of the first tissue area TA1 and/or of a further tissue area TA2, with the further tissue area TA2 bordering on the first tissue area TA1. In one embodiment, the model may further simulate a mechanical interaction between the first tissue area TA1 and the further tissue area TA2 SIM-WW.

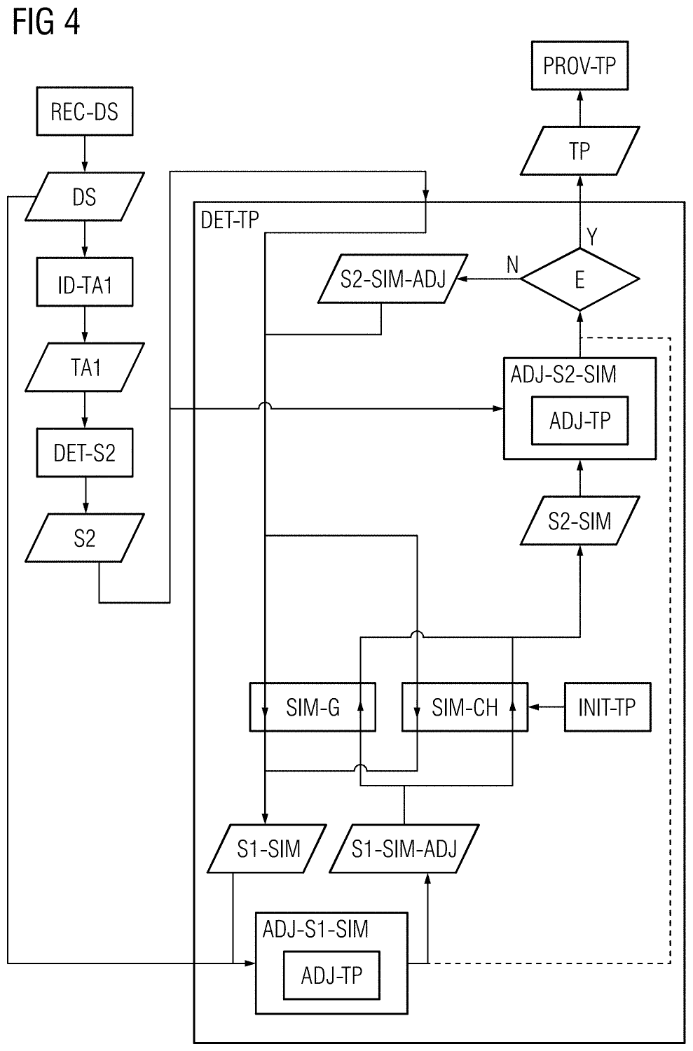

Shown schematically in FIG. 4 is a further embodiment of the method for provision PROV-TP of therapy plan TP. In this embodiment, the determination DET-TP of the therapy plan TP may include the acts a.1) to b.1). The method may start when first executed with the act a.1), for example, where the second state is predetermined as the second initial state. Further, a start value for the at least one parameter of the therapy plan TP may be received, geometrically predetermined, or initialized by a random seed generator INIT-TP. In act a.1), a first simulated state S1-SIM of the first tissue area TA1 may be determined for the at least one first point in time by inverse simulation of the biological growth SIM-G and of the change SIM-CH of the first tissue area TA1 between the at least one first point in time and the at least one second point in time, starting from the second initial state. In a further act a.2), the first simulated state S1-SIM may be adjusted ADJ-S1-SIM to the first state by adjustment ADJ-TP of the at least one parameter of the therapy plan TP. Further, the adjusted first simulated state S1-SIM-ADJ may be provided as the first initial state. Further, in a further act b.1), a second simulated state S2-SIM of the first tissue area TA1 may be determined for the at least one second point in time by simulation of the biological growth SIM-G and the change SIM-CH of the first tissue area TA1 between the at least one first point in time and the at least one second point in time, starting from the first initial state. In a further act b.2), the second simulated state S2-SIM may be adjusted ADJ-S2-SIM to the second state S2 by adjustment ADJ-TP of the at least one parameter of the therapy plan TP. Further, the adjusted second simulated state S2-SIM-ADJ may be provided as the second initial state. In one embodiment, the acts a.1) to b.2) may be carried out repeatedly Y until an abort condition E. In FIG. 4, the temporal direction (e.g., the inversion) of the simulation of the biological growth SIM-G and of the change SIM-CH of the first tissue area TA1 are illustrated by arrows pointing in different directions.

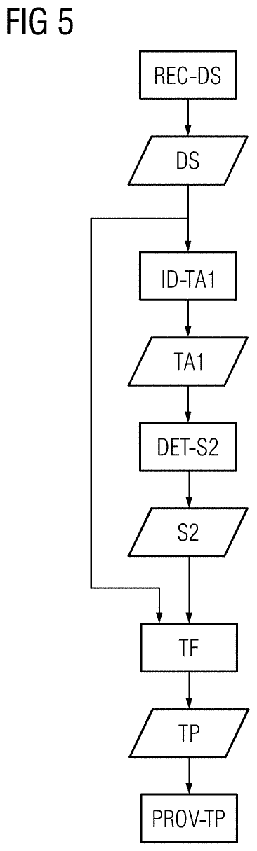

FIG. 5 shows a schematic diagram of a further embodiment of the method for provision PROV-TP of a therapy plan TP. In this embodiment, the model may include a trained function TF. At least one parameter of the trained function TF may further be adjusted by a comparison of a training therapy plan with a comparison therapy plan.

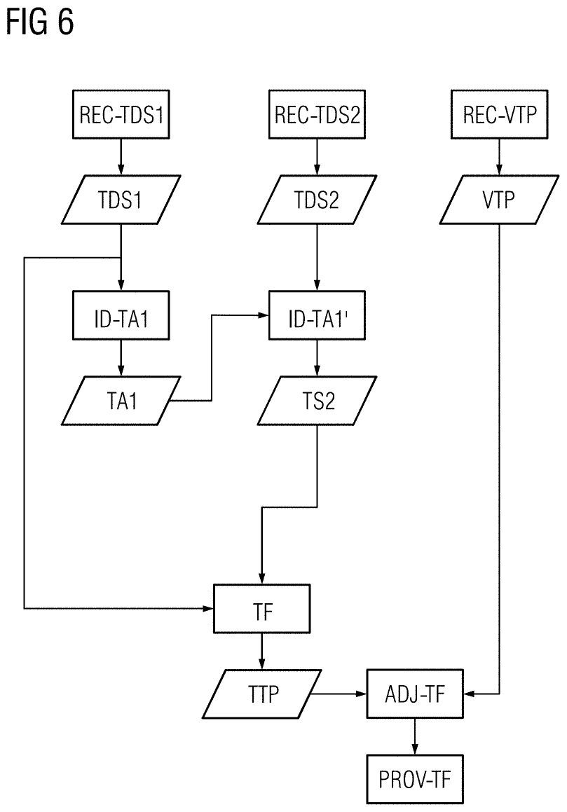

Shown schematically in FIG. 6 is an embodiment of a computer-implemented method for provision PROV-TF of a trained function TF. In a first act, a preacquired first training dataset TDS1 may be received REC-TDS1. In this case, the first training dataset TDS1 may map an examination object including a first tissue area TA1 at at least one first point in time. In a second act, a mapping of the first tissue area TA1 may be identified ID-TA1 in the first training dataset TDS1. In a third act, a preacquired second training dataset TDS2 may be received REC-TDS2. In this case, the second training dataset TDS2 may map the examination object including the first tissue area TA1 at at least one second point in time after the first point in time. In a fourth act, a further mapping of the first tissue area TA1 in the second training dataset TDS2 may be identified ID-TA1′ as the second training state TS2. In a fifth act, a comparison therapy plan VTP for a therapy apparatus may be received REC-VTP. In this case, the comparison therapy plan VTP may have at least one parameter for control of the therapy apparatus. Further, before the beginning of the method, between the at least one first point in time and the at least one second point in time, a change in the first tissue area TA1 may have been created by the therapy apparatus in accordance with the comparison therapy plan VTP. In a sixth act, a training therapy plan TTP for the therapy apparatus may be determined by applying the trained function TF to input data. In this case, the input data may be based on the first training dataset TDS1 and the second training state TS2. In a seventh act, at least one parameter of the trained function TF may be adjusted ADJ-TF by a comparison of the training therapy plan TTP with the comparison therapy plan VTP. Further, the training therapy plan TTP may be provided as output data of the trained function TF. In an eighth act, the trained function TF may be provided PROV-TF.

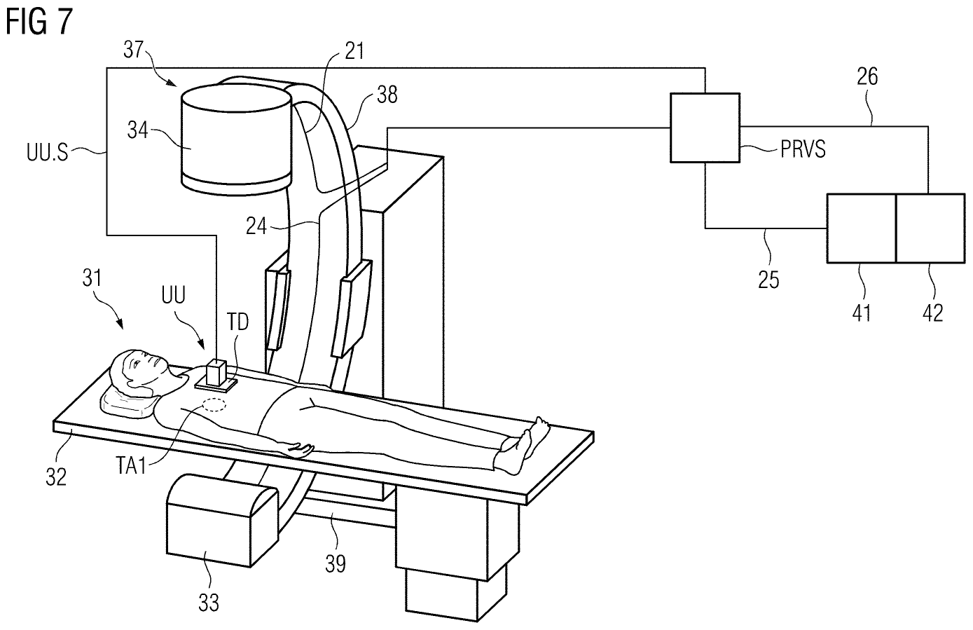

Shown schematically in FIG. 7 is an embodiment of a system. In this embodiment, the system may have a provision unit PRVS and a therapy apparatus. The therapy apparatus may further have an ultrasound unit UU. In this embodiment, the therapy apparatus may be embodied to create the change in the first tissue area TA1. The provision unit PRVS may further be configured to control the therapy apparatus (e.g., the ultrasound unit UU), in such a way with the aid of the therapy plan TP that the change is created between the at least one first point in time and the at least one second point in time in the first tissue area TA1. The provision unit PRVS may be embodied to control the therapy apparatus (e.g., the ultrasound unit UU) using a signal UU. S in accordance with the therapy plan TP.

In one embodiment, the ultrasound unit UU may have at least one transducer TD that is configured to emit the therapeutic ultrasound (e.g., an ultrasound field) in accordance with the therapy plan TP. Further, the ultrasound unit UU may have a coupling unit (not shown here), for example, with a tank that may be filled with a coupling medium (e.g., a liquid and/or a gel). The coupling unit may be configured to couple the at least one transducer TD to a surface of the examination object 31 acoustically. The at least one transducer TD (e.g., the coupling unit) may further be arranged on an examination object 31 (e.g., on a surface of the examination object 31). This enables the therapeutic ultrasound to be coupled through the surface of the examination object 31 (e.g., a skin surface) into the first tissue area TA1. In this case, the therapeutic ultrasound for this may be embodied to create at least one lesion in the first tissue area TA1 as the change. The examination object 31 may be arranged on a patient support apparatus 32.

The system may further have a medical C-arm x-ray device 37, for a medical imaging device for recording the dataset, for example. The medical C-arm x-ray device 37 may have a detector 34 (e.g., an x-ray detector) and an x-ray source 33. For recording the dataset, the arm 38 of the medical C-arm x-ray device 37 may be supported movably about one or more axes. The medical C-arm x-ray device 37 may further include a further movement unit 39 (e.g., a wheel system and/or rail system and/or a robot arm) that makes a movement of the medical C-arm x-ray device 37 in the room possible. The detector 34 and the x-ray source 34 may be fastened in a defined arrangement movably to a common C-arm 38.

To record the dataset of the examination object 31, the provision unit PRVS may send a signal 24 to the x-ray source 33. Thereafter, the x-ray source 33 may emit an x-ray bundle (e.g., a cone beam and/or fan beam and/or parallel beam). When the x-ray bundle (e.g., after an interaction with the examination object 31, such as the first tissue area TA1) strikes a surface of the detector 34, the detector 34 may send a signal 21 to the provision unit PRVS. The provision unit PRVS may receive the dataset with the aid of the signal 21.

The system may further include an input unit 42 (e.g., a keyboard) and/or a display unit 41 (e.g., a monitor and/or display). The input unit 42 may be integrated into the display unit 41 (e.g., with a capacitive and/or resistive input display). The input unit 41 may further be embodied for acquiring an input of a medical operator. For this, the input unit 42 may, for example, send a signal 26 to the provision unit PRVS. The display unit 41 may further be configured to display a graphical display of the dataset and/or of the therapy plan. For this, the provision unit PRVS may send a signal 25 to the display unit 41, for example.

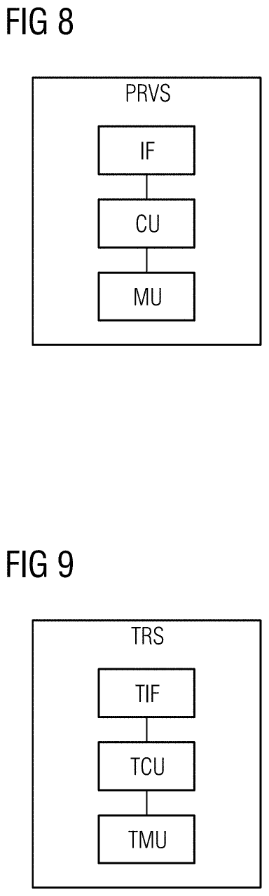

Shown schematically in FIG. 8 is one embodiment of a provision unit PRVS. In this case, the provision unit PRVS may include an interface IF, a computing unit CU, and a memory unit MU. The provision unit PRVS may be configured to carry out a method for provision of a therapy plan PROV-TP and its aspects, in that the interface IF, the computing unit CU, and the memory unit CU are configured to carry out the corresponding method acts.

FIG. 9 shows a schematic diagram of one embodiment of a training unit TRS. The training unit TRS may include a training interface TIF, a training memory unit TMU, and a training computing unit TCU. The training unit TRS may be configured to carry out a method for provision of a trained function PROV-TF and its aspects, in that the training interface TIF, the training memory unit TMU, and the training computing unit TCU are configured to carry out the corresponding method acts.

The provision unit PRVS and/or the training unit TRS may, for example, involve a computer, a microcontroller, or an integrated circuit. As an alternative, the provision unit PRVS and/or the training unit TRS may involve a real or virtual network of computers (a technical term for a real network is Cluster, a technical term for a virtual network is Cloud). The provision unit PRVS and/or the training unit TRS may also be configured as a virtual system that is executed on a real computer or a real or virtual network of computers (e.g., virtualization).

An interface IF and/or a training interface TIF may involve a hardware or software interface (e.g., PCI bus, USB, or Firewire). A computing unit CU and/or a training computing unit TCU may have hardware elements or software elements (e.g., a microprocessor or a Field Programmable Gate Array (FPGA)). A memory unit MU and/or a training memory unit TMU may be realized as volatile memory (e.g., Random Access Memory (RAM)) or as permanent mass storage (e.g., hard disk, USB stick, SD card, Solid State Disk).

The interface IF and/or the training interface TIF may, for example, include a number of sub-interfaces that carry out different acts of the respective method. In other words, the interface IF and/or the training interface TIF may also be interpreted as a plurality of interfaces IF or a plurality of training interfaces TIF. The computing unit CU and/or the training computing unit TCU may, for example, include a number of sub-computing units that carry out different acts of the respective method. In other words, the computing unit CU and/or the training computing unit TCU may also be interpreted as a plurality of computing units CU or a plurality of training computing units.

The schematic diagrams shown in the described figures do not in any way depict a scale or a measure of size.

The methods and apparatuses described above in detail merely involve exemplary embodiments that may be modified by the person skilled in the art in a wide diversity of ways without departing from the field of the invention. Further, the use of the indefinite article “a” or “an” does not exclude the features concerned also being able to be present multiple times. Likewise, the terms “unit” and “element” do not exclude the components concerned consisting of a number of interacting subcomponents, which, where necessary, may also be spatially distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for provision of a therapy plan, the method comprising:
receiving a dataset that is preacquired, wherein the dataset maps an examination object including a first tissue area at at least one first timepoint;
identifying a mapping of the first tissue area in the dataset as a first state;
specifying a second state for the first tissue area, wherein the second state defines a spatial extent, a second tissue parameter of at least a part of the first tissue area at at least one second timepoint, or a combination thereof, the at least one second timepoint being after the at least one first point in time;
determining a therapy plan for a therapy apparatus by application of a model to input data, wherein the input data is based on the dataset and the second state,
wherein the therapy plan has at least one parameter for control of the therapy apparatus, wherein a change in the first tissue area between the at least one first timepoint and the at least one second timepoint is createable through the control of the therapy apparatus in accordance with the therapy plan, wherein the model simulates a biological growth and the change in the first tissue area between the at least one first timepoint and the at least one second timepoint, and wherein the model maps the first state to the second state, the second state to the first state, or a combination thereof; and
providing the therapy plan as output data of the model.

2. The method of claim 1, wherein the spatial extent is a maximum spatial extent.

3. The method of claim 1, wherein the dataset has at least one first tissue parameter of the first tissue area, a further tissue area, or the first tissue area and the further tissue area,
wherein the further tissue area borders on the first tissue area.

4. The method of claim 3, wherein the model further simulates a mechanical interaction between the first tissue area and the further tissue area.

5. The method of claim 1, wherein identifying the mapping of the first tissue area comprises identifying a mapping of at least one critical structure, an edge area, or the at least one critical structure and the edge area of the first tissue area in the dataset, and
wherein the model leaves out the at least one critical structure, the edge area, or the at least one critical structure and the edge area in the simulation of the change in the first tissue area.

6. The method of claim 1, wherein the second state defines a value, a range of values, or the value and the range of values for the spatial extent, the second tissue parameter of the at least one part of the first tissue area for the at least one second timepoint, a period of time comprising a number of second timepoints, up to a temporal maximum value, or comprising the number of second timepoints and up to a temporal maximum value, or any combination thereof.

7. The method of claim 6, wherein the first tissue area, in at least one area, borders on a further tissue area, and
wherein the second state defines the value, the range of values, or the value and the range of values for the spatial extent, the second tissue parameter at least for the border, or a combination thereof.

8. The method of claim 1, wherein, during the control of the therapy apparatus in accordance with the therapy plan, at least one lesion is creatable in the first tissue area,
wherein the model simulates a reduction in the spatial extent of the first tissue area, a change in the second tissue parameter as the change between the at least one first timepoint and the at least one second timepoint, or a combination thereof, and
wherein the simulation of the change comprises a simulation of a transporting away of lysed tissue from the at least one lesion.

9. The method of claim 8, wherein the transporting is physiological transporting.

10. The method of claim 8, wherein the therapy apparatus has an ultrasound unit,
wherein therapeutic ultrasound is emittable by the ultrasound unit during the control of the therapy apparatus in accordance with the therapy plan into the first tissue area,
wherein the at least one parameter predetermines a frequency, a bandwidth, an amplitude, a phase, a pulse duration, a focus position, a beam alignment, a beam shaping, a duty cycle, or any combination thereof of the therapeutic ultrasound, such that the at least one lesion is creatable in the first tissue area by the therapeutic ultrasound.

11. The method of claim 1, wherein determining the therapy plan comprises:
determining a first simulated state of the first tissue area for the at least one first timepoint, the determining of the first simulated state comprising inverse simulating the biological growth and the change in the first tissue area between the at least one first timepoint and the at least one second timepoint, starting from a second initial state;
adjusting the first simulated state to the first state, the adjusting of the first simulated state comprising adjusting the at least one parameter of the therapy plan and providing the adjusted first simulated state as the first initial state;
determining a second simulated state of the first tissue area for the at least one second timepoint, the determining of the second simulated state comprising simulating the biological growth and the change in the first tissue area between the at least one first timepoint and the at least one second timepoint, starting from the first initial state;
adjusting the second simulated state to the second state, the adjusting of the second simulated state comprising adjusting the at least one parameter of the therapy plan and providing the adjusted second simulated state as the second initial state,
wherein the determining of the first simulated state, the adjusting of the first simulated state, the determining of the second simulated state, and the adjusting of the second simulated state are carried out repeatedly up to occurrence of an abort condition, and
wherein, when the method is carried out for the first time:
the method starts with the determining of the first simulated state and the second state is predetermined as the second initial state, or the method starts with the determining of the second simulated state, and the first state is predetermined as the first initial state; and
a start value is received for the at least one parameter of the therapy plan, the start value is geometrically predetermined, or the start value is initialized by a random seed generator.

12. The method of claim 1, wherein the model comprises a trained function,
wherein the method further comprises adjusting at least one parameter of the trained function by a comparison of a training therapy plan with a comparison therapy plan.

13. The method of claim 1, wherein the dataset maps an initial change in the first tissue area,
wherein the initial change has been created before a beginning of the method by the therapy apparatus in accordance with an initial therapy plan, and
wherein the dataset also has the initial therapy plan.

14. The method of claim 1, wherein providing the therapy plan comprises transmitting the at least one parameter to the therapy apparatus, performing a preparatory adjustment of at least one operating parameter, at least one positioning parameter, or the at least one operating parameter and the at least one positioning parameter to the therapy apparatus in accordance with the therapy plan, or a combination thereof.

15. The method of claim 14, wherein the transmitting, the performing, or the transmitting and the performing are without the control of the therapy apparatus.

16. A computer-implemented method for provision of a trained function, the computer-implemented method comprising:
receiving a first training dataset that is preacquired, wherein the first training dataset maps an examination object including a first tissue area at at least one first timepoint;
identifying a mapping of the first tissue area in the first training dataset;
receiving a second training dataset that is preacquired, wherein the second training dataset maps the examination object including the first tissue area at at least one second timepoint after the first timepoint;
identifying a further mapping of the first tissue area in the second training dataset as the second training state;
receiving a comparison therapy plan for a therapy apparatus, wherein the comparison therapy plan has at least one parameter for control of the therapy apparatus,
wherein, before the beginning of the computer-implemented method, between the at least one first timepoint and the at least one second timepoint, a change in the first tissue area has been created by the therapy apparatus in accordance with the comparison therapy plan;
determining a training therapy plan for the therapy apparatus, the determining of the training therapy plan comprising applying the trained function to input data, wherein the input data is based on the first training dataset and the second training state, wherein the training therapy plan is provided as output data of the trained function;
adjusting at least one parameter of the trained function, the adjusting of the at least one parameter of the trained function comprising comparing the training therapy plan with the comparison therapy plan; and
providing the trained function.

17. A provision unit for provision of a therapy plan, the provision unit comprising:
a processor configured to:
receive a dataset that is preacquired, wherein the dataset maps an examination object including a first tissue area at at least one first timepoint;
identify a mapping of the first tissue area in the dataset as a first state;
specify a second state for the first tissue area, wherein the second state defines a spatial extent, a second tissue parameter of at least a part of the first tissue area at at least one second timepoint, or a combination thereof, the at least one second timepoint being after the at least one first point in time;
determine a therapy plan for a therapy apparatus by application of a model to input data, wherein the input data is based on the dataset and the second state,
wherein the therapy plan has at least one parameter for control of the therapy apparatus, wherein a change in the first tissue area between the at least one first timepoint and the at least one second timepoint is createable through the control of the therapy apparatus in accordance with the therapy plan, wherein the model simulates a biological growth and the change in the first tissue area between the at least one first timepoint and the at least one second timepoint, and wherein the model maps the first state to the second state, the second state to the first state, or a combination thereof; and
provide the therapy plan as output data of the model.

18. A system comprising:
a provision unit for provision of a therapy plan, the provision unit comprising:
a processor configured to:
receive a dataset that is preacquired, wherein the dataset maps an examination object including a first tissue area at at least one first timepoint;
identify a mapping of the first tissue area in the dataset as a first state;
specify a second state for the first tissue area, wherein the second state defines a spatial extent, a second tissue parameter of at least a part of the first tissue area at at least one second timepoint, or a combination thereof, the at least one second timepoint being after the at least one first point in time;
determine a therapy plan for a therapy apparatus by application of a model to input data, wherein the input data is based on the dataset and the second state, wherein the therapy plan has at least one parameter for control of the therapy apparatus, wherein a change in the first tissue area between the at least one first timepoint and the at least one second timepoint is createable through the control of the therapy apparatus in accordance with the therapy plan, wherein the model simulates a biological growth and the change in the first tissue area between the at least one first timepoint and the at least one second timepoint, and wherein the model maps the first state to the second state, the second state to the first state, or a combination thereof; and
provide the therapy plan as output data of the model; and
a therapy apparatus, wherein the therapy apparatus is configured to create the change in the first tissue area,
wherein the provision unit is configured to control the therapy apparatus with the aid of the therapy plan such that the change is created in the first tissue area between the at least one first point in time and the at least one second point in time.

19. The system of claim 18, wherein the therapy apparatus includes an ultrasound unit,
wherein the ultrasound unit is configured to send therapeutic ultrasound into the first tissue area, and
wherein the therapeutic ultrasound is configured to create at least one lesion in the first tissue area as the change.

20. The system of claim 18, further comprising a medical imaging device,
wherein the medical imaging device is configured to record, provide, or record and provide the dataset.
